(19) United States
(12) Patent Application Publication
(10) Pub. No.: US 2024/0009379 A1
(43) Pub. Date: Jan. 11, 2024

(54) CONTINUOUS FLUID IRRIGATION ASSEMBLY

(71) Applicant: CREATIVE MEDICAL SOLUTIONS INC., Toronto (CA)

(72) Inventors: Brian CARILLO, Toronto (CA); Monica FARCAS, Toronto (CA); Ray Alwin Perera JAYATUNGA, Toronto (CA)

(21) Appl. No.: 18/190,106

(22) PCT Filed: Sep. 28, 2021
(86) PCT No.: PCT/IB2021/058811

§ 371 Date: Mar. 26, 2023

Related U.S. Application Data
(60) Provisional application No. 63/084,866, filed on Sep. 29, 2020.

(51) Int. Cl. A61M 3/02

(52) U.S. Cl. CPC A61M 3/022; A61M 3/0202; A61M 3/0233; A61M 2205/3334

(57) ABSTRACT

A method for continuous fluid irrigation comprising generating a first and a second output signal based on a volume of fluid within a first irrigation bag and a volume of fluid within a second irrigation bag; and moving a first switching device from a first position to a second position based on at least one of the first or the second output signal, wherein in the first position, free flow is allowed through the first irrigation tubing, and in the second position, free flow is allowed through the second irrigation tubing.

### FIELD OF THE DISCLOSURE

This disclosure relates to continuous fluid irrigation systems and in particular a continuous fluid irrigation assembly that automatically switches between irrigation bags responsive to a predetermined condition.

### BACKGROUND

Continuous fluid irrigation plays an indispensable role in many minimally invasive surgical procedures, especially those that use an endoscopic or arthroscopic approach. Fluid irrigation into the body cavity generates pressure which is needed to distend the cavity and increases the size of the operative field as well as establishes homeostasis through the tamponading of small venous vessels. Concurrently, movement of excess fluid out of the operative field helps to remove blood and debris, allowing for optimal visualization as well as dissipation of heat generated by surgical instruments.

Intra-operatively, optimal irrigation is defined as a stable state of irrigation that is capable of providing positive intra-cavity pressure while maintaining sufficient flow. Excessive flow and pressure may lead to tissue distortion and fluid extravasation while insufficient flow may lead to collapse of the operative space.

Various irrigation systems are currently available and are used in a surgical setting, with the gravity-fed irrigation system being the most common due to its safety, simplicity and low cost. However, one frequent problem with the gravity-fed irrigation system described above is the abrupt loss of entry pressure and flow when the irrigation bags become empty, thus making it necessary to temporarily halt the surgery until the irrigation bag is replaced.

Hematuria (blood in the urine) is a common condition associated with multiple possible factors including: infection, carcinoma, prostatic enlargement, pelvic radiation, and post-operatively from transurethral resections of the bladder or prostate. This condition can potentially lead to clot urinary retention (inability to urinate due to obstruction of the urinary system by blood clots). This is a very painful condition that requires manual bladder irrigation (most commonly performed in the emergency room or the ward without anaesthetic). In order to prevent clot retention, hematuria is commonly managed in hospital with continuous bladder irrigation (CBI). A three-way catheter is inserted in the bladder and continuous flow of normal saline irrigation is maintained in and out of the bladder to prevent clot formation during times of active bleeding. The rate of the inflow of the fluid (provided through the gravity-fed irrigation system described above) is visually titrated by nurses by assessing the effluent colour (if clear, slow down or stop the irrigation, if very hematuric increase the inflow). Currently, the irrigation inflow rate is adjusted using a simple roller-ball type device along the tubing.

In both scenarios, the responsibility of changing and monitoring the irrigation bags, as well as titrating the inflow, falls upon nurses. Operating room nurses must glance at the irrigation bags every few minutes to either note the fluid level or switch the bag, while ward and ER nurses must keep track of multiple patients and remember to go and physically check that a bag has not run dry on a patient running CBI. This practice is both time consuming and also draws the nursing staff's attention from other important duties.

In order to minimize the risks of irrigation interruption for these patients, there is a clear need for a device that can automate the monitoring, changing, and flow titration of the irrigation bags. Others have attempted to solve this issue by developing a protocol that involves hanging irrigation bags at different levels to allow continuous flow to be maintained between the two bags naturally. However, these solutions only serve to increase the time between each bag and do not truly automate the process of exchanging irrigation bags.

It would be advantageous to provide a device that monitors the volume of irrigation fluid used and automatically switch between irrigation bags.

### SUMMARY

A continuous fluid irrigation assembly for use with at least a first irrigation bag operably attached to a first irrigation tubing and a second irrigation bag operably attached to a second irrigation tubing, the continuous fluid irrigation assembly comprising: a first bag sensor operably attached to the first irrigation bag, wherein the first bag sensor generates a first output signal based on a volume of fluid within the first irrigation bag; a second bag sensor operably attached to the second irrigation bag, wherein the second bag sensor generates a second output signal based on a volume of fluid within the second irrigation bag; a first switching device attached to the first and the second irrigation tubing, the first switching device having at least a first position and a second position, wherein in the first position, free flow is allowed through the first irrigation tubing, in the second position, free flow is allowed through the second irrigation tubing, the first switching device is communicatively coupled to the first and the second bag sensor, and the first switching device moves from the first position to the second position based on at least one of the first and the second output signal.

A flow rate control module for use in association with irrigation tubing, comprising a variable switching device that has a plurality of different positions between a first position and a second position whereby in the first position flow through the irrigation tubing is stopped, and in the second position free flow is allowed through the irrigation tubing.

A method for continuous fluid irrigation comprising generating a first and a second output signal based on a volume of fluid within a first irrigation bag and a volume of fluid within a second irrigation bag; and moving a first switching device from a first position to a second position based on at least one of the first or the second output signal, wherein in the first position, free flow is allowed through the first irrigation tubing, and in the second position, free flow is allowed through the second irrigation tubing.

A continuous fluid irrigation assembly for use with a plurality of bags, wherein each of the plurality of bags is attached to one of a plurality of tubings, the continuous fluid irrigation assembly comprising: one of a plurality of bag sensors operably attached to the first irrigation bag, wherein each of the plurality of bag sensors generates a corresponding output signal based on a volume of fluid within the corresponding irrigation bag; a switching device attached to the plurality of tubings, wherein the switching device has a plurality of positions, wherein in each of the plurality of positions, free flow is allowed through one of the plurality of tubings, the switching device is communicatively coupled to the plurality of bag sensors, and the switching device moves to one of the plurality of positions based on at least one of the plurality of output signals.

Further features will be described or will become apparent in the course of the following detailed description.

### BRIEF DESCRIPTION OF THE DRAWINGS

The embodiments will now be described by way of example only, with reference to the accompanying drawings, in which:

FIG. 1 is a schematic view of a continuous bladder irrigation system using two irrigation bags;

FIG. 2 is a schematic view of a continuous bladder irrigation system similar to that shown in FIG. 1 but using four irrigation bags;

FIG. 3 is a schematic view of a continuous bladder irrigation system similar to that shown in FIG. 2 but also including an effluent sensing device and a fluid irrigation rate controller;

FIG. 4A is an enlarged perspective view of the fluid switching device shown in FIGS. 1 to 3 as viewed from the back;

FIG. 4B is a perspective view of the fluid switching device shown in FIGS. 1 to 3 as viewed from the side back;

FIG. 4C is a perspective view of the fluid switching device shown in FIGS. 1 to 3 as viewed from the side front;

FIG. 5 is a blown apart perspective view of the fluid switching device shown in FIGS. 4A, 4B and 4C;

FIG. 6A is a perspective view of the effluent sensing device shown in FIG. 3 showing the door open;

FIG. 6B is a perspective view of the effluent sensing device shown in FIG. 3 showing the door closed;

FIG. 7 is blown apart perspective view of the effluent sensing device shown in FIG. 6A and FIG. 6B;

FIG. 8A is a perspective view of the continuous bladder irrigation rate controller shown in FIG. 3 as viewed from one side and shown open;

FIG. 8B is a perspective view of the continuous bladder irrigation rate controller shown in FIG. 3 as viewed from the other side and shown open;

FIG. 8C is a side view of continuous bladder irrigation rate controller shown in FIG. 3 and shown open;

FIG. 9 is a blown apart perspective view of the continuous bladder irrigation rate controller shown in FIGS. 8A, 8B and 8C;

FIG. 10A is the beginning of a process flow diagram showing the main components of the four irrigation bag continuous bladder irrigation system shown in FIG. 2;

FIG. 10B is the continuation of FIG. 10A; and

FIG. 10C is the further continuation of FIG. 10A and FIG. 10B.

### DETAILED DESCRIPTION

The Figures are not to scale and some features may be exaggerated or minimized to show details of particular elements while related elements may have been eliminated to prevent obscuring novel aspects. Therefore, specific structural and functional details disclosed herein are not to be interpreted as limiting but merely as a basis for the claims and as a representative basis for teaching one skilled in the art to variously employ the present invention. For purposes of teaching and not limitation, the illustrated embodiments are directed to continuous fluid irrigation assemblies.

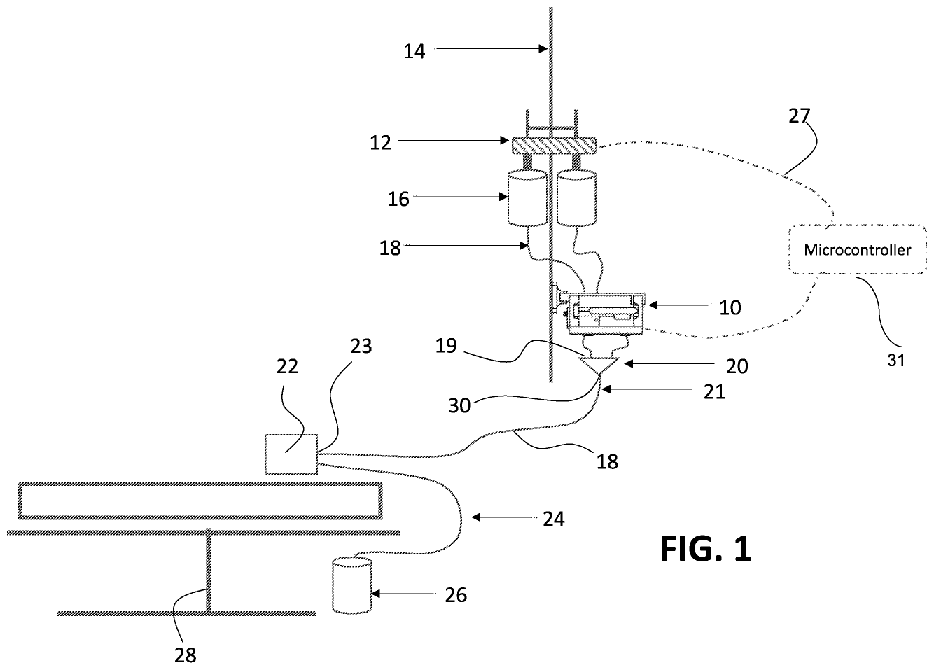

FIGS. 1 to 3 show alternate continuous fluid irrigation assemblies. The continuous fluid irrigation assemblies shown herein are for use in the operating room, in a patient's hospital room, or in the emergency room.

Referring to FIG. 1 the continuous fluid irrigation assembly includes a switching device 10 operably connected to a bag sensor 12. The bag sensor 12 may be a weight sensor. The continuous fluid irrigation assembly may be used with a conventional IV (intravenous) or irrigation pole 14 which typically can hold/accommodate a plurality of irrigation bags 16. In this embodiment there are two irrigation bags 16. Irrigation tubing 18 extends from each bag to a Y connector 20 through the switching device 10. The Y connector 20 has two inlets or inputs 19 and one outlet or output 21. Tubing 18 connects the outlet 21 of the Y connector 20 to the inlet 23 of a medical instrument 22. The medical instrument 22 may be a catheter or other surgical tool. The medical instrument 22 releases irrigation fluid into the patient cavity. A drainage tube 24 is used to drain fluid from the patient cavity. The drainage tube 24 is operably connected to a collection bag 26. The microcontroller 31 attached to switching device 10 has an operable connection 27 to bag sensors 12 so that it can decide when to switch bags. The microcontroller 31 may be directly or wirelessly connected 27 to the bag sensors 12. The sensors 12 provide fluid volume information, taken to be the volume of fluid present in the irrigation bags, directly to the microcontroller 31. More specifically, the sensors 12 preferably relay weight information to the microcontroller, which can be configured to determine the volume of fluid present in the irrigation bags. In the embodiment described herein the sensors 12 are weight sensors and from the weight information the volume of the fluid may be determined. The bag sensors 12 may be load cells and the load cells are operably connected to a microcontroller 31.

Based on defined threshold levels, the microcontroller 31 instructs switching device 10 to alternate between two configurations in order to change the irrigation fluid source to the full bag. Position feedback from switching device 10 is relayed to the microcontroller 31 via an inbuilt potentiometer present in the motor. The defined threshold levels may be predetermined or adjustable. Typically, the IV or irrigation pole 14 is situated proximate to the patient's bed or operating room table 28.

Typically, a connector assembly 30 includes two pieces of tubing 18 connected to the inlet 19 of Y connector 20 and one piece of tubing 18 connected to the outlet 21 of the Y connector 20. The connector assembly 30 may be purchased as a sterile unit. The switching device 10 may be attached to the two pieces of tubing 18 attached to the inlet 19 of the Y connector 20 without affecting the sterility of the connector assembly 30.

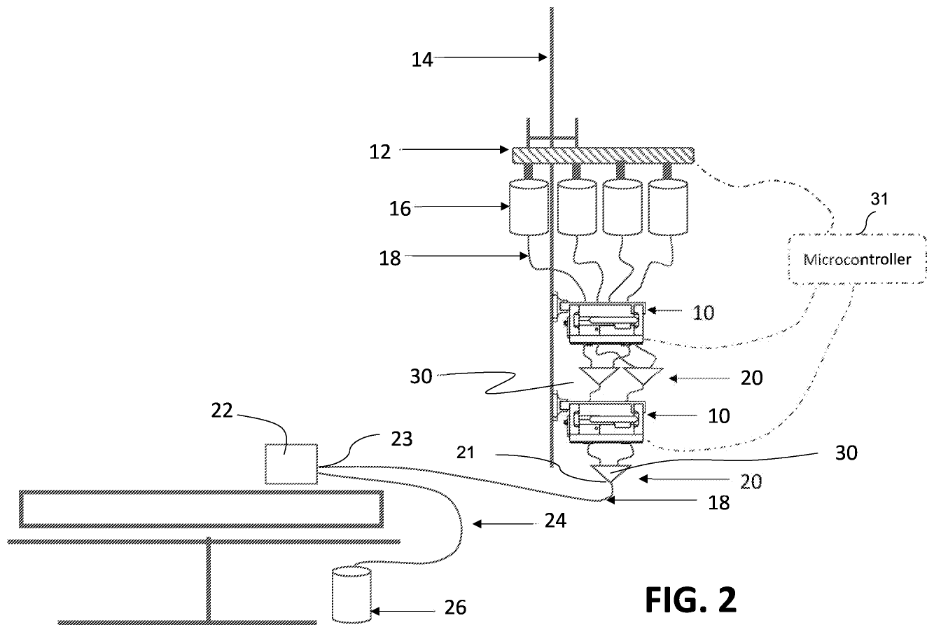

The embodiment shown in FIG. 2 is similar to that shown in FIG. 1 but using four irrigation bags 16, two switching devices 10, and three connector assemblies 30. The connector assemblies 30 are arranged such that there are two upper Y connectors 20 and one lower connector 20. As with the embodiment shown in FIG. 1, tubing 18 extend from each bag to an upper Y connector 20 through the first switching device 10. There are two upper Y connectors 20 operably connected to the four irrigation bags 16 via tubing 18. Tubing 18 is connected to the output 19 of the upper Y connector 20 through a second switching device 10 to a single lower Y connector 20. As described above tubing 18 to be attached to inlet 23 of the medical instrument 22 is connected to the output 21 of Y connector 20.

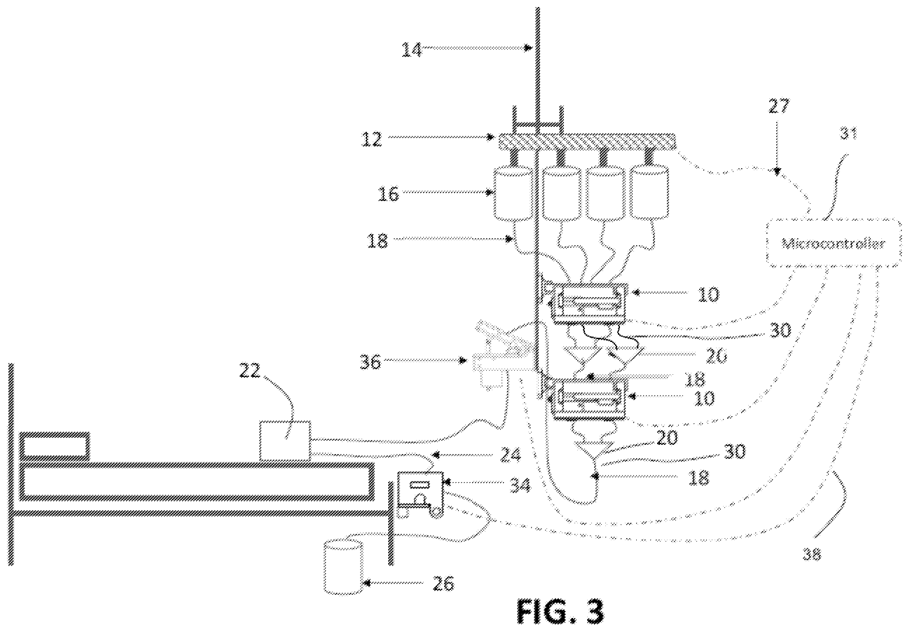

The embodiment shown in FIG. 3 is similar to that shown in FIG. 2 but further includes an effluent sensing device 34 operably connected to a continuous bladder irrigation (CBI) rate controller 36 through the microcontroller. The effluent sensing device 34 is operably connected 38 through the microcontroller 31 to the CBI rate controller 36 to provide feedback. It may be directly connected 38 or wirelessly connected thereto. The effluent sensing device is connected to the bladder drainage tube 24 as is described in more detail below.

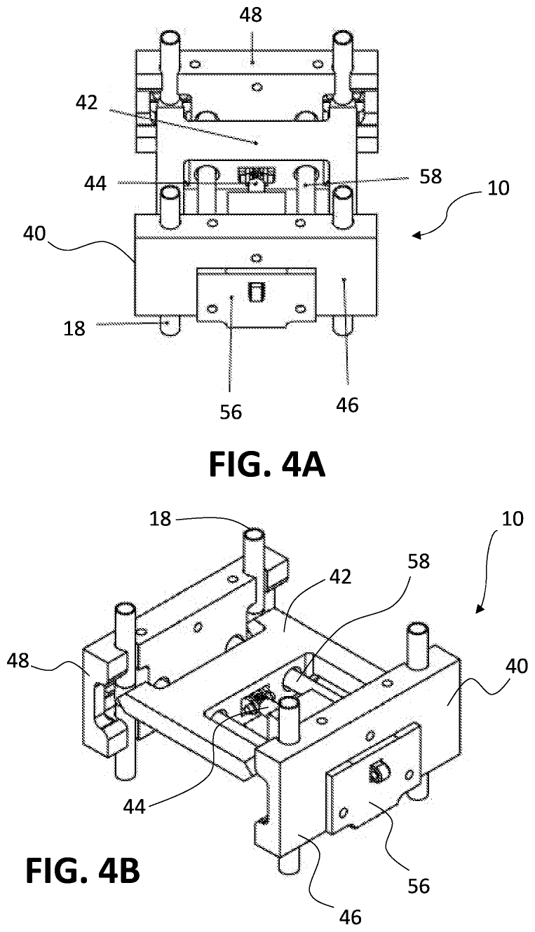

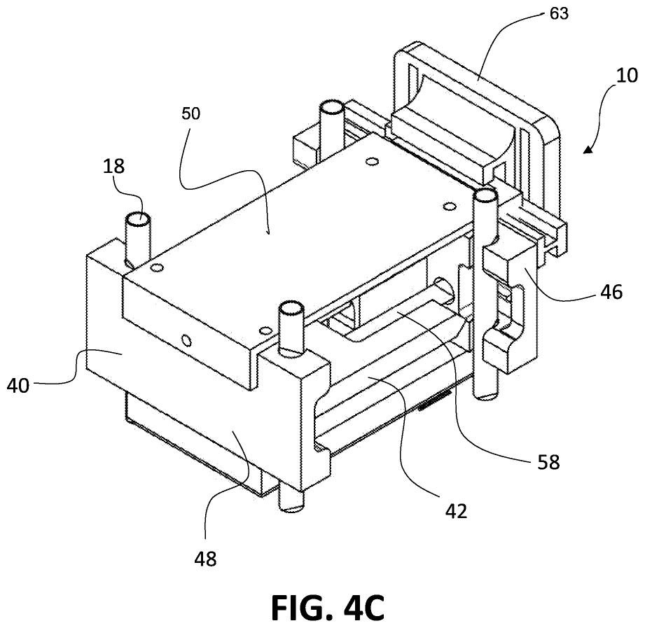

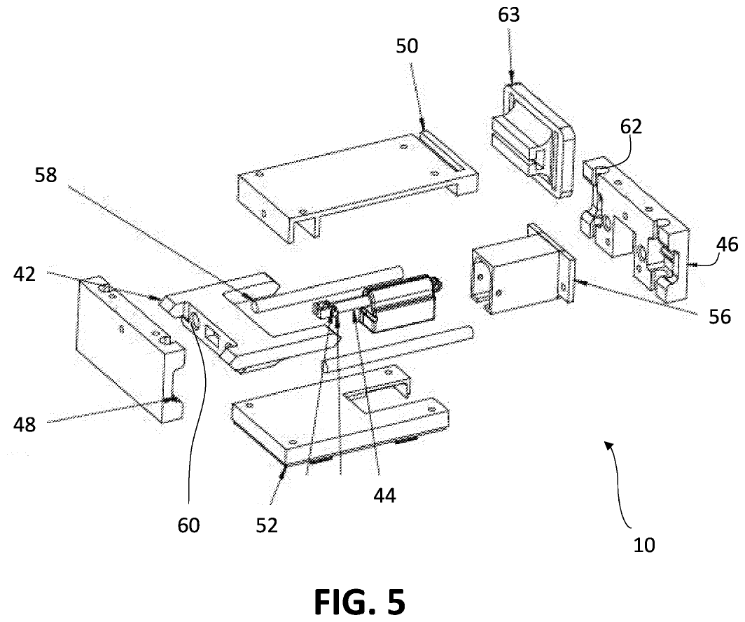

As depicted in FIG. 4A, FIG. 4B, FIG. 4C and FIG. 5 the switching device 10 includes a housing 40, a pinch arm 42 and an actuator 44. The housing 40 includes a front pinch plate 48, a back pinch plate 46, a top plate 50 and a bottom plate 52. The actuator 44 is operably connected to the housing 40. The actuator 44 has an actuator cover 56 which is operably connected to the housing 40. A pair of guide rods 58 extend between the front pinch plate 48 and the back pinch plate 46. The pinch arm 42 has a pair of guide rod holes and the pinch arm slides along the guide rods 58. The pinch arm 42 is attached to the actuator 44. The actuator 44 moves the pinch arm 42 between the front pinch plate 48 and the back pinch plate 46. The front plate 48 and the back plate 46 have irrigation tube guides 62 formed therein for receiving the irrigation tube 18. The pinch arm 42 moves between a first position wherein the pinch arm 42 pinches the irrigation tube 18 between one of the front pinch plate 48 and the back pinch plate 46 and the pinch arm 42, and a second position wherein the pinch arm 42 pinches the irrigation tube 18 between the other of the front pinch plate 48 and the back pinch plate 46 and the pinch arm 42. Each time the pinch arm pinches an irrigation tube, it stops flow within the irrigation tube. A pole mount 63 is operably attached to the housing 40. The pole mount 63 is configured to be attachable to an IV pole.

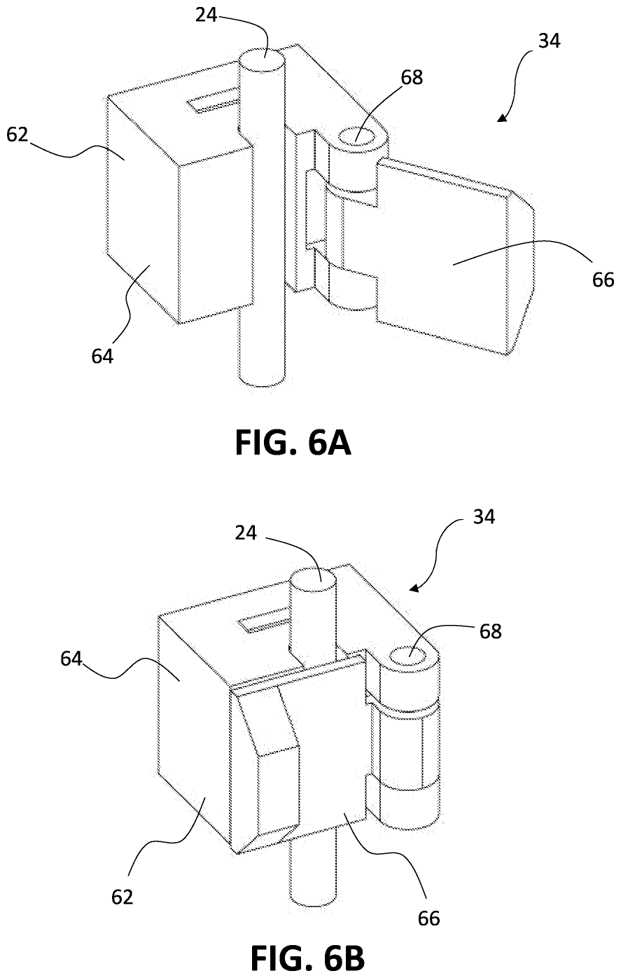

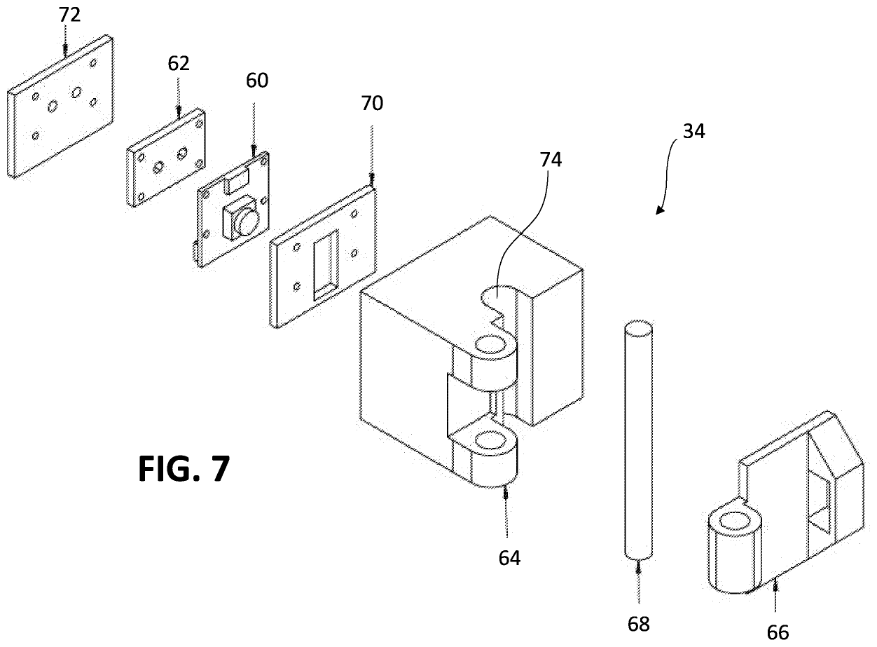

The microcontroller 31 is operably attached to the actuator 44. In response to bag weight information from the weight sensors 12, the microcontroller 31 determines which position the pinch arm 42 must assume and activates the actuator 44 accordingly. On activation the pinch arm 42 toggles between the first position and the second position. With reference to FIG. 6A, FIG. 6B and FIG. 7, The effluent sensing device 34 includes a camera and a camera housing 62. The camera housing 62 includes a camera block 64 with a block door 66 hingeably attached thereto with a hinge pin 68. A camera cover assembly 70 holds the camera 60 in the camera block 64. A back plate 72 is attached to the camera block 64. The camera block 64 has a groove 74 formed therein for receiving drainage tubing 24 as shown in FIG. 6A and FIG. 6B.

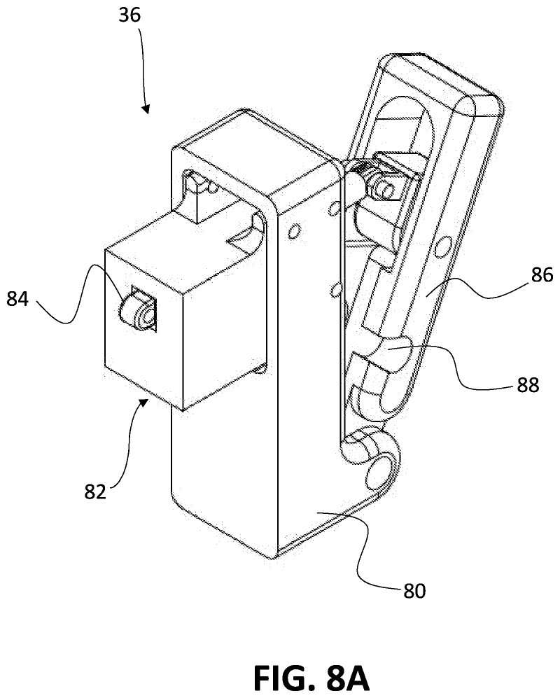

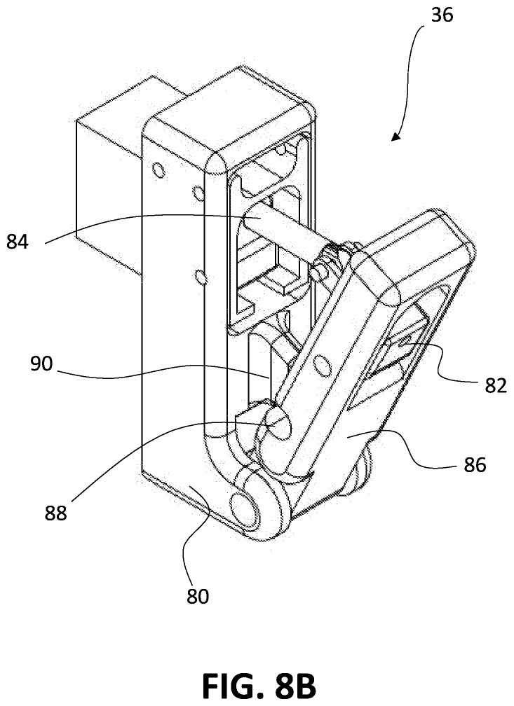

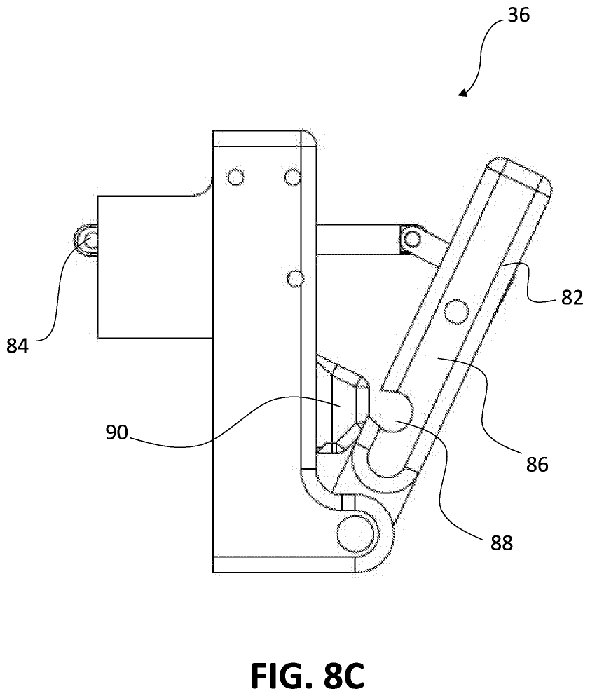

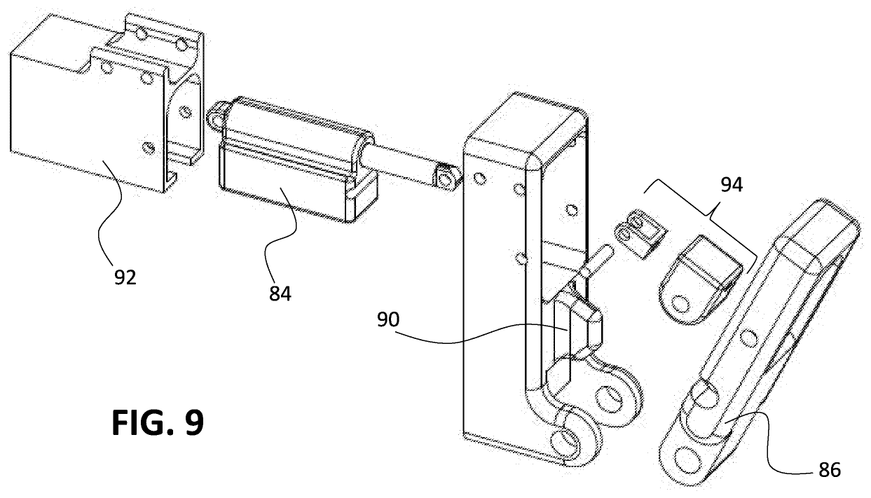

FIG. 8A to FIG. 8C and FIG. 9 show an embodiment of continuous bladder irrigation (CBI) rate controller 36. CBI rate controller 36 is mechanically similar to switching device 10 except that it is a variable switching device and has multiple positions for the pinch arm 42 between a first position and a second position. The variable switching device or CBI rate controller 36 has a plurality of different positions between a first position wherein it pinches the irrigation tube and stops flow therein to the second position allowing free flow thereout thereby varying the flow. The position is set responsive to signals from the microcontroller 31 attached to the effluent sensing device 34. CBI rate controller 36 includes a housing 80 for receiving tubing 18 and a pinch mechanism 82. The pinch mechanism 82 includes an actuator 84 operably attached to a pinch lever arm 86 that hingeably moves inwardly and outwardly to selectively pinch the tube. The housing 80 has a guide 88 formed therein for receiving the tubing. A post 90 is positioned such that as the pinch lever arm 86 moves inwardly the tubing is pushed against the post 90 and flow in the tubing is restricted. The housing 80 includes an actuator cover 92. The actuator 84 is hingeably attached to the lever arm 86 with connectors 94.

Drainage tube 24 passes through effluent sensing device 34 which measures blood concentration in the effluent. In the embodiment shown herein the drainage tubing 24 is standard urinary drainage tubing. This information is passed to the microcontroller 31. If the microcontroller 31 determines that the blood concentration is above a predetermined threshold, it adjusts the graduated pinch mechanism 82 of the rate controller 36 to allow irrigation fluid to flow faster. Note in the embodiment shown herein the microcontroller 31 controls both the CBI rate controller 36 and the switching device 10, however each device may have a dedicated microcontroller. Alternately, if blood concentration is below a predetermined threshold, it slows down the fluid rate. A measurement, or set of measurements, is taken at predetermined intervals. In one example, a measurement is taken every minute and the position of the pinch lever arm 86 is varied based on the measurement thereby adjusting the flow based on blood concentration.

It will be appreciated that there are a number of different ways in which to determine the blood concentration or an approximation of the blood concentration. In the example shown herein the camera 60 determines the blood concentration indirectly by determining the pixel colour density. The effluent sensing device 34 captures an image of the fluid inside the bladder drainage tube 24. The pixel information is processed by a microcontroller 31 and red, green and blue channel information is calculated. The red, green and blue channel information of each pixel of the total image is then averaged. The level of bleeding is then determined by calculating the total red channel values as a proportion of the total red, green, blue channel information. That is the level of bleeding=red/(red+green+blue). The higher the value of red pixel information in the picture taken by the effluent sensing device 34, the greater the level of bleeding is determined to be, and the greater the opening of the pinch lever arm 86. The position of the variable switching device or continuous bladder irrigation rate controller 36 is chosen responsive to the level of bleeding.

While the above describes embodiments where a camera is used, one of skill in the art would appreciate that any suitable image capture or image sensor device can be used to determine blood concentration.

Other examples of methods or devices to determine blood concentration either directly or indirectly could include colour sensors, pulse oximeters, transparency sensors, transmittance sensors or spectrometers.

In the two bag system of FIG. 1, before starting all of the bags are clipped. To start with, fluid draining from the first bag the pinch arm 42 starts in the first position wherein the pinch arm 42 pinches an irrigation tube 18 attached to the second bag between the pinch arm 42 and the front pinch plate 48. An irrigation tube 18 attached to the first bag is on the other side of the pinch arm such that when the pinch arm moves to the second position that irrigation tube is pinched between the back plate 48 and the pinch arm 42. When in the second position the irrigation tube from the first bag is pinched and fluid from the first bag is not draining. Therefore, when the first bag sensor senses that the bag weight is below a predetermined value the pinch arm 42 toggles from the first position to the second position. In the second position the irrigation tube attached to the first bag is pinched and the irrigation tube attached to the second bag is no longer pinched and thus fluid flows therefrom. The empty bag in the first bag position may be changed.

When the system senses that the second bag is below a predetermined weight the pinch arm 42 toggles between the second position and the first position. Where the first bag has been replaced then fluid can then drain from the first bag and so on. In this manner, the pinch arm 42 toggles between the first position and the second position in order to maintain a continuous flow of irrigation from a non-empty irrigation bag source. Alternatively, if the first bag has not been replaced, the microcontroller 31 will sense that both bags are below a predetermined level and the pinch arm will move to a middle position where neither irrigation tubes are pinched. In some embodiments, the microcontroller 31 will switch to the bag having the lowest level. In yet other embodiments, the microcontroller will switch to the bag having the highest level.

In the event where no signals are being received from the bag weight sensors 12, the pinch arm will move to a middle position where neither irrigation tubes are pinched. This will prevent a scenario where no irrigation fluid is draining.

In the four bag system of FIG. 2 the pinch arm 42 of an upper switch device 10 starts in the first position wherein the pinch arm 42 pinches an irrigation tube 18 attached to a first bag and another irrigation tube attached to the third bag is pinched between the pinch arm 42 and the front pinch plate 48. An irrigation tube 18 attached to the second bag and another irrigation tube attached to the fourth bag is on the other side of the pinch arm 42 such that when the pinch arm 42 moves to the second position the irrigation tubes are pinched between the back plate 46 and the pinch arm 42. When the pinch arm 42 is in the first position, fluid flows from the second and fourth bags and when the pinch arm 42 is in the second position, irrigation fluid flows from the first and third bags. The irrigation tubes from the first bag and the second bag are connected to a first upper Y connector 20 and the irrigation tubes attached to the third bag and the fourth bag are connected to the second upper Y connector When the pinch arm 42 is in the first position fluid flows from the second and fourth bags and when the pinch arm 42 is in the second position fluid flows from the first and third bags. The irrigation tubes attached to the output from the upper Y connectors are connected to a lower Y connector and are positioned in a lower switch device 10. The irrigation tubes are arranged such that is pinched in the first position and the other tube is pinched in the second position.

The irrigation tubes are arranged such that when the upper switch device is in the second position and the lower switch device is in the second position, fluid is flowing from only the first bag, whilst the other three bags are prevented from draining. When the upper switch device is in the first position and the lower switch device is in the second position fluid is flowing from the second bag. When the upper switch device is in the second position and the lower switch device is in the first position fluid is flowing from the third bag. When the upper switch device is in the first position and the lower switch device is in the first position fluid is flowing from the fourth bag. In this way, through the combination of configurations of the upper and lower switching devices, the source of irrigation can be selected from any one of the four irrigation bags.

The microcontroller 31 waits until the currently active bag weight is below a predetermined threshold weight in order to select which bag should be active next (has fluid or not). It then sends signal to switch the appropriate pinch system.

In one embodiment, the switch device 10 is provided with a back up battery. Thereby the device is effectively a standard two bag system and therefore it will require manual changing of bags immediately as they run out.

There are 2 control systems:
1. The controller monitoring the bags and actuating the pinch mechanisms. In the embodiment shown herein the controller is external. Alternatively, the controller may be encased in or internal to the switch device 10.
2. The controller monitoring the camera/blood sensor. In the embodiment shown herein the controller is external. Alternatively, the controller may be encased in or internal to the CBI rate controller 36.

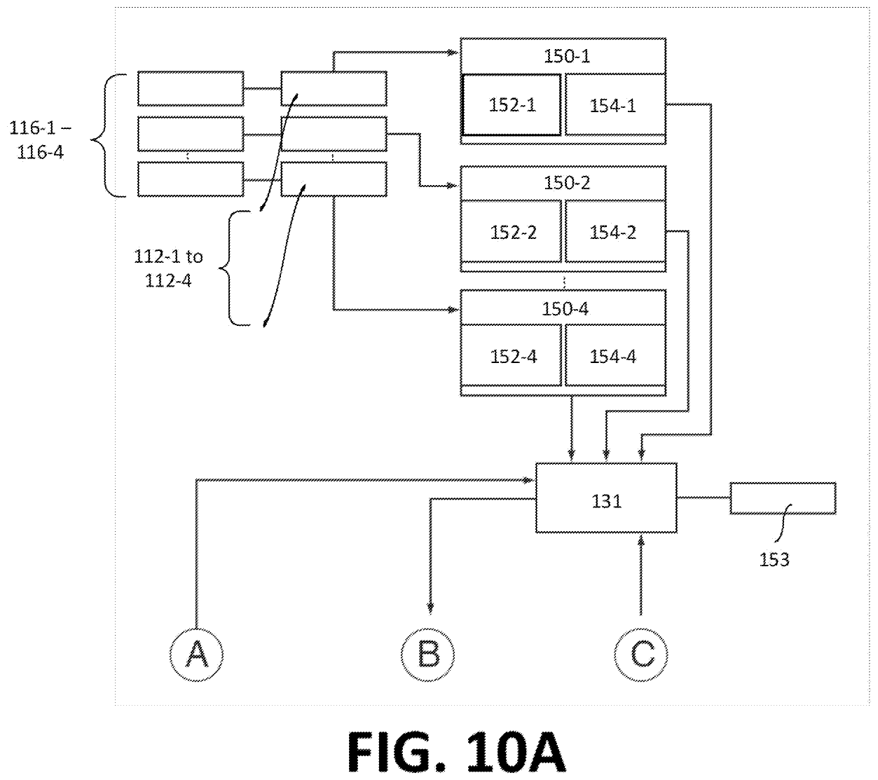

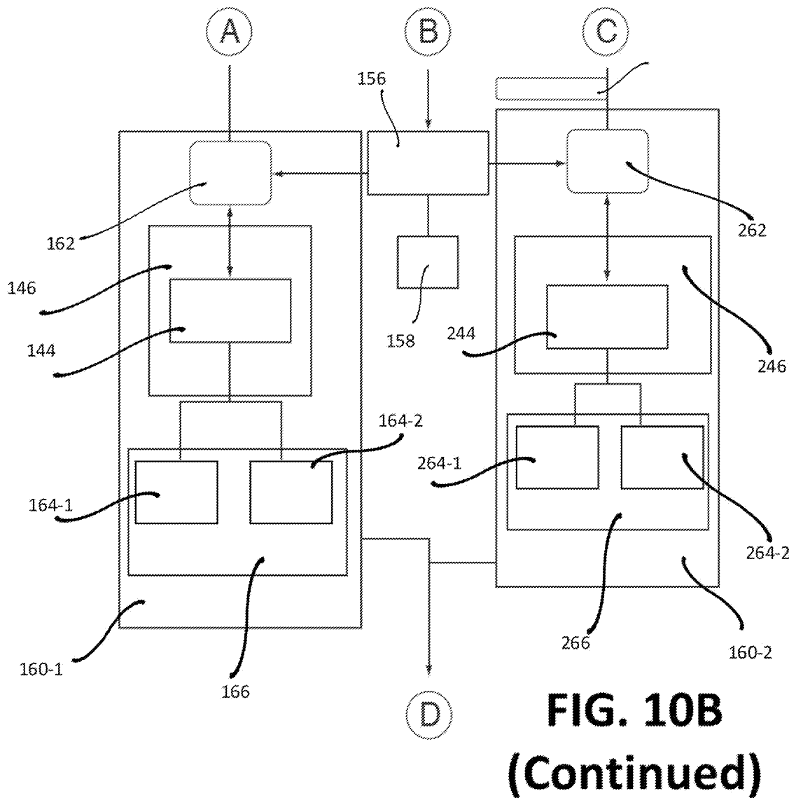

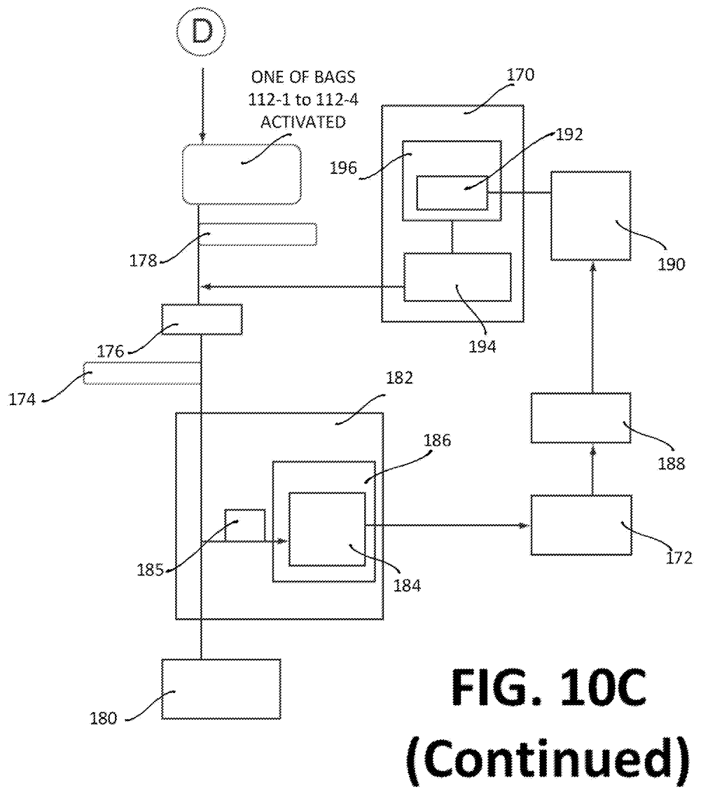

More specifically, the first control system is composed of a programmable microcontroller as shown in FIG. 10A. The microcontroller receives the fluid weight information from the load cells and determines whether the fluid is above or below a predetermined threshold. In response to registering an empty bag, the microcontroller is programmed to deliver a signal to drive the pinch arm to the correct position via activation of the actuator. Position feedback of the pinch arm is relayed to the microcontroller via inbuilt potentiometers in the actuator. The flow diagram shown in FIG. 10A, continued on FIG. 10B and further continued on FIG. 10C shows this in more detail.

In FIG. 10A, bag sensors such as load cells 112-1-112-4 are similar to, for example, bag sensors or load cells 12 of FIG. 3. Irrigation bags 116-1 to 116-4 correspond to, for example, the first, second, third and fourth irrigation bags 16 of FIG. 3. Load cells 112-1 measures the weight of corresponding irrigation bag 116-1, load cell 112-2 measures the weight of corresponding irrigation bag 116-2 and so on. The output signals generated by load cells 112-1 to 112-4 are transmitted to corresponding interface modules 150-1 to 150-4. The interface modules 150-1 to 150-4 act to interface load cells 112-1 to 112-4 to microcontroller 131.

Each of interface modules 150-1 to 150-4 comprise instrumentation amplifiers 152-1 to 152-4 and analog-to-digital (A/D) converters 154-1 to 154-4. The analog-formatted output signals from load cells 112-1 to 112-4 are transmitted to instrumentation amplifiers 152-1 to 152-4. The amplified analog-formatted output signals are transmitted to A/D converters 154-1 to 154-4, where these signals converted into digital-formatted signals. The digital-formatted output signals are then transmitted from interface modules 150-1 to 150-4 to microcontroller 131. The microcontroller 131 is powered by a power supply such as 5V power supply 153. As explained above, microcontroller 131 receives the amplified digital-formatted signals and performs calculations described previously to determine whether the fluid is above or below a predetermined threshold. Based on this determination, the microcontroller produces an output signal.

Referring to FIG. 10B, the output signal from microcontroller 131 is transmitted to dual DC motor driver 156, which is powered by a power supply such as 12V DC power supply 158. Dual DC motor driver 156 is coupled to subsystems 160-1 and 160-2.

Subsystem 160-1 comprises actuator 144 and actuator cable adapter 162. Similar to as previously explained, subsystem 160-1 plays a similar role as the first switching device 10 in FIG. 3, that is, it controls the inputs to the upper Y connectors. Actuator 144 is similar to actuator 44 as described in FIGS. 4A, 4B, 4C and 5. Dual DC motor driver 156 is coupled to actuator cable adapter 162 which is in turn coupled to actuator 144. Actuator 144 is housed in actuator shell 146, which is similar to housing 40 in FIGS. 4A, 4B, 4C and 5.

Similar to the previously described explanation of the working of actuator 44, actuator 144 controls a pinch mechanism 166 which can occupy either a first position 164-1 or a second position 164-2. Then, dual DC motor driver 156 outputs a control signal to actuator 144 via adapter cable 162 so that pinch mechanism 166 either occupies position 164-1 or 164-2. The control signal outputted by dual DC motor driver 156 is generated based on the output signal from microcontroller 131 transmitted to dual DC motor driver 156. As explained above, inbuilt potentiometers within actuator 144 relay position feedback via adapter cable 162 to microcontroller 131.

As explained previously, in the first position 164-1, irrigation tubes coupled to IV bags 112-1 and 112-3 are pinched. In the second position 164-2, irrigation tubes coupled to irrigation bags 112-2 and 112-4 are pinched. Similar to as explained previously, the irrigation tubes coupled to IV bags 112-1 and 112-2 are input to one of the upper Y connectors, and irrigation bags 112-3 and 112-4 are input to another of the upper Y connectors.

Subsystem 160-2 comprises actuator 244 and actuator cable adapter 262. Similar to as previously explained, subsystem 160-2 plays a similar role as the second switching device 10 in FIG. 3, that is, it controls the inputs to the lower Y connector. Dual DC motor driver 156 is coupled to actuator cable adapter 262 which is in turn coupled to actuator 244. As explained previously, actuator 244 controls pinch mechanism 266 which can occupy a first position 264-1 or 264-2. As explained above, inbuilt potentiometers within actuator 244 relay position feedback via adapter cable 262 to microcontroller 131. Actuator 244 is housed in actuator shell 246.

Then, when the pinch mechanism 266 in subsystem 160-2 is in the first position 264-1, the output tube from the upper Y connector where tubes connected to irrigation bags 112-1 and 112-2 are input to is pinched. When the pinch mechanism is in the second position 264-2, the output tube from the upper Y connector where tubes connected to irrigation bags 112-3 and 112-4 are input to is pinched.

The following example is illustrative. When pinch mechanism 166 occupies position 164-1, the tubes coupled to irrigation bags 112-1 and 112-3 are pinched, while free flow is allowed through the tubes coupled to irrigation bags 112-2 and 112-4. Then, when pinch mechanism 266 occupies position 264-1:
the output tube from the upper Y connector where tubes connected to irrigation bags 112-1 and 112-2 are input to is pinched;
the tubes coupled to irrigation bags 112-1 and 112-3 are also pinched;
therefore irrigation bag 112-4 is activated as free flow is allowed through:
   1. the tube attached to irrigation bag 112-4, and
   2. the output tube from the upper Y-connector where the tube attached to irrigation bag 112-4 is input to.

One of skill in the art would see that by using an appropriate combination of the positions in pinch mechanisms in subsystems 160-1 and 160-2, each of the four bags 112-1 to 112-4 can be activated as is shown in FIG. 10C.

Referring to FIG. 10C, irrigation fluid 178 flows through the output tube which is operably attached to an activated bag to patient 176. The flow rate of irrigation fluid 178 is controlled by flow rate control module 170, which is similar to CBI rate controller 36. Similar to the above explanation for CBI rate controller 36, flow rate control module 170 is a variable switching device with a graduated pinch mechanism 194. Similar to as explained above, the graduated pinch mechanism 194 has a plurality of positions between a first position, wherein it pinches an irrigation tube and stops flow therein, and a second position, which allows free flow through the irrigation tube. The graduated pinch mechanism 194 thereby controls the flow of irrigation fluid through the output irrigation tube.

As explained above, the position of the graduated pinch mechanism is set responsive to signals from microcontroller 172. Similar to the above explanation, effluent or waste fluid 174 from the patient within a drainage tube such as the previously mentioned drainage tube 24 is imaged within irrigation tube scanner module 182, while en route to collection bag 180. irrigation tube scanner module 182 is similar to effluent sensing device 34, and comprises a blood concentration measurement device as previously mentioned. In the illustrated embodiment, the blood concentration measurement device is camera module 184 residing within camera shell 186. Camera module 184 is similar to camera 60, and camera shell 186 is similar to camera housing 62. Then, similar to as explained above, waste fluid 174 is imaged 185 by camera module 184. The output image from camera module 184 is transmitted to microcontroller 172. Similar to as previously explained, the output image is processed by microcontroller 172 to determine the blood concentration level. As explained above, in some embodiments pixel colour density is used to determine the blood concentration level. Then, the higher the value of red pixel information in the image 185 taken by camera module 184, then the greater the blood concentration level is determined to be by microcontroller 172. Then, microcontroller 172 will send a signal to DC motor driver 188 to increase the flow rate of irrigation fluid 178 to patient 176. Dual DC motor driver 188 is communicatively coupled to actuator 192. An illustrative embodiment is shown in FIG. 10C, whereby dual DC motor driver 188 is communicatively coupled to actuator 192 via actuator cable adaptor 190. Then, dual DC motor driver 188 sends a signal via control actuator cable adapter 190 to actuator 192. Actuator 192 controls graduated pinch mechanism 194, which in turn controls the flow rate of irrigation fluid 178 to the patient 176 by selecting one of the plurality of positions as mentioned above. Therefore, similar to as explained above, the graduated pinch mechanism 194 within flow rate module 170 is adjusted by dual DC motor driver 188 responsive to the blood concentration. As also explained above, other examples of blood concentration measurement devices which measure blood concentration either directly or indirectly include image sensors, image capture devices, colour sensors, pulse oximeters, transparency sensors, transmittance sensors or spectrometers.

The above details 2 control systems with 2 separate microcontrollers 131 and 172, one used to monitor the bags and actuate the pinch mechanisms and the other used to monitor the blood concentration and control irrigation fluid flow to the patient via flow rate module 170 or CBI rate controller 36. It will be appreciated by those skilled in the art that one control system may control both the switching device 10 and the CBI rate controller 36. For example, one microcontroller can be used to control both the switching devices 10 and the CBI rate controller 36. An example of this is shown in FIG. 3, where microcontroller 31 controls both switching devices 10 and the CBI rate controller 36.

While the above has been described for irrigation fluids, one of skill in the art would know that the above described embodiments can be used for other fluids and bags, such as intravenous (IV) fluid bags.

The above presents embodiments having switching arrangements with two stages of switching wherein each stage has one switching device. Other arrangements are possible. In some embodiments the two stages of switching described above are integrated into a single stage comprising one or more switching devices. In these embodiments, the one or more switching devices allow free flow from only one of a plurality of operably attached bags, where each of the plurality of operably attached bags is attached to one of a plurality of tubings. Each of the plurality of irrigation bags is operably attached to one of a plurality of bag sensors communicatively coupled to a microcontroller, as described above. Each of the plurality of bag sensors generates one of a plurality of output signals. The one or more switching devices have a plurality of positions. In each of the plurality of positions, free flow is allowed through one of the plurality of tubings attached to one of the plurality of bags. To enable the one or more switching devices to move to one of the plurality of positions: The plurality of output signals generated by the plurality of bag sensors are received by the microcontroller similar to as described above. The microcontroller then performs processing of one or more of the received plurality of output signals, similar to as described above. The microcontroller is communicatively coupled to the one or more switching devices, as described above. Based on this processing, the microcontroller generates and transmits instructions to the one or more switching devices to move to one of the plurality of positions, also similar to as described above.

In some of these embodiments, the single stage comprises the flow rate module or CBI rate controller described above, so as to vary the flow rate through the selected one of the plurality of tubings. The flow rate module or CBI rate controller is, for example, implemented within the one or more switching devices.

Furthermore, while the above presents embodiments where a flow merging device such as a Y connector having two inputs and one output is utilized, one of skill in the art would know that there are other types of flow merging devices which can be used. These flow merging devices have more than two inputs and a single output.

The microcontroller as presented above can be implemented in a variety of ways. In some embodiments, the microcontroller comprises, for example, a laptop, tablet, smartphone, or any appropriate computing device. In yet other embodiments, the microcontroller is implemented using hardware, software, or a combination of hardware and software. In other embodiments, the microcontroller is coupled to one or more external systems. These external systems can be used for functions such as alerting, inventory management and patient management.

Generally speaking, the systems described herein are directed to a continuous fluid irrigation assembly. Various embodiments and aspects of the disclosure are described in the detailed description. The description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components.

As used herein the “operably connected” or “operably attached” means that the two elements are connected or attached either directly or indirectly. Accordingly, the items need not be directly connected or attached but may have other items connected or attached therebetween.

1. A continuous fluid irrigation assembly for use with at least a first irrigation bag operably attached to a first irrigation tubing and a second irrigation bag operably attached to a second irrigation tubing, the continuous fluid irrigation assembly comprising:
a first bag sensor operably attached to the first irrigation bag, wherein
the first bag sensor generates a first output signal based on a volume of fluid within the first irrigation bag;
a second bag sensor operably attached to the second irrigation bag, wherein
the second bag sensor generates a second output signal based on a volume of fluid within the second irrigation bag;
a first switching device attached to the first and the second irrigation tubing,
the first switching device having at least a first position and a second position, wherein
in the first position, free flow is allowed through the first irrigation tubing,
in the second position, free flow is allowed through the second irrigation tubing,
the first switching device is communicatively coupled to the first and the second bag sensor, and
the first switching device moves from the first position to the second position based on at least one of the first and the second output signal.

2. The continuous fluid irrigation assembly of claim 1, wherein
the microcontroller is communicatively coupled to the first switching device, the first bag sensor and the second bag sensor;
the first and the second output signal are received by a microcontroller;
the microcontroller processes at least one of the first and the second output signal; and
the microcontroller controls the first switching device to move from the first position to the second position based on the processing.

3. The continuous fluid irrigation assembly of claim 1 wherein the first output signal is sent from the first bag sensor when a weight of the first irrigation bag drops below a level.

4. The continuous fluid irrigation assembly of claim 1, wherein
the assembly is for use with a third irrigation bag attached to a third irrigation tubing and a fourth irrigation bag attached to a fourth irrigation tubing;
the assembly comprises
a third bag sensor operably attached to the third irrigation bag, wherein
the third bag sensor generates a third output signal based on a volume of fluid within the third irrigation bag, and
a fourth bag sensor operably attached to the fourth irrigation bag, wherein
the fourth bag sensor generates a fourth output signal based on a volume of fluid within the fourth irrigation bag; and
the first switching device is attached to the third and the fourth irrigation tubing, wherein
in the first position,
free flow is allowed through the first and third irrigation tubing, in the second position,
free flow is allowed through the second and fourth irrigation tubing,
the first switching device is communicatively coupled to the third and fourth bag sensor, and
the first switching device moves from the first position to the second position based on at least one of the first, second, third and fourth output signal.

5. The continuous fluid irrigation assembly of claim 4, wherein
the assembly includes a second switching device;
the first and the second irrigation tubing are connected to inputs of a first Y connector after passing through the first switching device;
the third and fourth irrigation tubing are connected to inputs of a second Y connector after passing through the first switching device;
output irrigation tubings from the first and second Y connectors are attached to the second switching device;
the second switching device having a first position and a second position, wherein
in the first position, free flow is allowed through the output irrigation tubing from the first Y connector,
in the second position, free flow is allowed through the output irrigation tubing from the second Y connector,
the second switching device is communicatively coupled to the first, second, third and fourth bag sensors, and
the second switching device moves from the first position to the second position based on at least one of the first, second, third or fourth output signals.

6. The continuous fluid irrigation assembly of claim 4, wherein
the microcontroller is communicatively coupled to the first switching device, the first bag sensor, the second bag sensor, the third bag sensor and the fourth bag sensor;
the first, second, third and fourth output signals are received by a microcontroller;
the microcontroller processes the first, second, third and fourth output signal; and
the microcontroller controls the first switching device to move from the first position to the second position based on the processing.

7. The continuous fluid irrigation assembly of claim 6, wherein
the microcontroller is communicatively coupled to the second switching device; and
the microcontroller controls the second switching device to move from the first position to the second position based on the processing.

8. The continuous fluid irrigation assembly of claim 5 wherein
the output irrigation tubing from the first and the second Y connectors are connected to inputs to a third Y connector after passing through the second switching device; and
the output irrigation tubing from the third Y connector is attached to a continuous bladder irrigation rate controller.

9. The continuous fluid irrigation assembly of claim 8 wherein:
the continuous bladder irrigation rate controller is a variable switching device having a plurality of different positions between
a first position wherein the variable switching device stops flow through the output irrigation tubing from the third Y connector, and
a second position wherein the variable switching device allows free flow through the output irrigation tubing from the third Y connector.

10. The continuous fluid irrigation assembly of claim 9 wherein
the variable switching device is communicatively coupled to an effluent sensing device; and
the variable switching device moves to one of the plurality of positions based on a signal generated by the effluent sensing device, thereby varying the flow through the output irrigation tubing.

11. The continuous fluid irrigation assembly of claim 10 wherein the effluent sensing device comprises a blood concentration measuring device.

12. The continuous fluid irrigation assembly of claim 11 wherein
the assembly comprises a microcontroller communicatively coupled to the effluent sensing device and the variable switching device;
the effluent sensing device generates the signal based on the blood concentration determined by the blood concentration measuring device;
the signal is processed by a microcontroller; and
the microcontroller controls the variable switching device to move to one of the plurality of positions based on the processing by the microcontroller.

13. A flow rate control module for use in association with irrigation tubing, comprising
a variable switching device that has a plurality of different positions between a first position and a second position whereby
in the first position flow through the irrigation tubing is stopped, and
in the second position free flow is allowed through the irrigation tubing.

14. The flow rate control module as claimed in claim 13 wherein:
the variable switching device is communicatively coupled to an effluent sensing device; and
the variable switching device moves to one of the plurality of positions based on a signal generated by the effluent sensing device.

15. The flow rate control module as claimed in claim 14 wherein:
a microcontroller is communicatively coupled to the effluent sensing device and the variable switching device;
the effluent sensing device comprises a blood concentration measuring device;
the effluent sensing device generates the signal based on the blood concentration determined by the blood concentration measuring device;
the signal is processed by a microcontroller; and
the microcontroller controls the variable switching device to move to one of the plurality of positions based on the processing by the microcontroller.

16. The flow rate control module of claim 15 wherein the blood concentration measuring device comprises one of:
a camera,
a camera module,
a colour sensor,
a pulse oximeter,
a transparency sensor,
a transmittance sensor, and
a spectrometer.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. A continuous fluid irrigation assembly for use with a plurality of bags, wherein each of the plurality of bags is attached to one of a plurality of tubings, the continuous fluid irrigation assembly comprising:
one of a plurality of bag sensors operably attached to the first irrigation bag, wherein
each of the plurality of bag sensors generates a corresponding output signal based on a volume of fluid within the corresponding irrigation bag;
one or more switching devices attached to the plurality of tubings, wherein
the one or more switching devices have a plurality of positions, wherein in each of the plurality of positions, free flow is allowed through one of the plurality of tubings,
the one or more switching devices are communicatively coupled to the plurality of bag sensors, and
the one or more switching devices move to one of the plurality of positions based on at least one of the plurality of output signals.

33. The assembly of claim 32, wherein the switching device comprises a flow rate module to vary the flow rate through the one of the plurality of tubings where free flow is allowed.
